(19) United States
(12) Patent Application Publication
(10) Pub. No.: US 2006/0106357 A1
(43) Pub. Date: May 18, 2006

(54) A method using a portable opaque re-closable container for disposal of used feminine hygiene products.

(76) Inventor: Rita C. McLean, Garland, TX (US)

Correspondence Address: RITA MCLEAN, 1825 LEWIS DRIVE, GARLAND, TX, 75041, US

(21) Appl. No.: 10/906,383

(22) Filed: Feb. 17, 2005

Related U.S. Application Data
(60) Provisional application No. 60/627,617, filed on Nov. 15, 2004.

(51) Int. Cl. A61F 13/15

(52) U.S. Cl. 604/385.02; 604/385.13

(57) ABSTRACT

A method to use an individual container made of resin material, opaque in color and leak-proof, used to dispose of used feminine hygiene products in a private manner. The container has two horizontal dimensions and two vertical dimensions. The two vertical dimensions and the bottom horizontal dimension are permanently sealed. The reclosable top horizontal dimension opens to insert the used feminine hygiene product inside the container. Then the container is closed. Once closed, the container is odor proof, leak-proof and the inside contents are not visible from an outside view of the container. The container is portable and convenient for the user to transport.

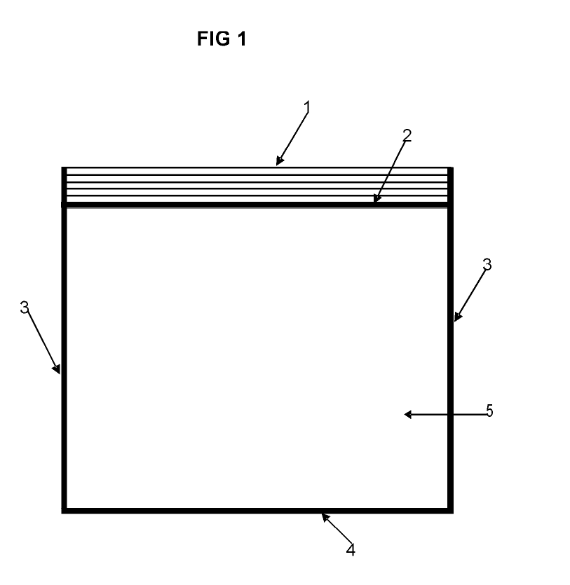

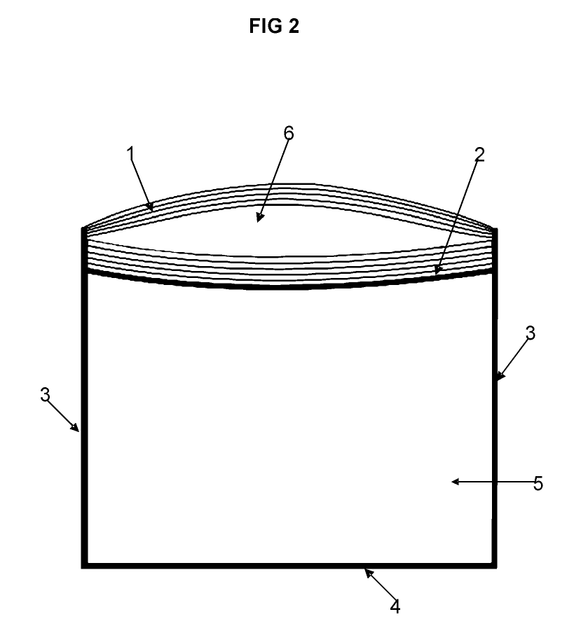

### BACKGROUND OF INVENTION

This invention pertains to the field of feminine hygiene products. This invention relates generally to using a portable opaque reclosable container made of a resin material to dispose of used feminine hygiene products. More specifically the invention is a method in which females use the portable container to dispose of used feminine hygiene products such as napkins and tampons. There are several containers and methods for disposing of used feminine hygiene products. However none appear to meet the characteristics described in this invention. The following are a few examples of U.S. patents which have been issued for disposing of used feminine hygiene products: U.S. Pat. Nos. 6,739,114; 6,120,743; 6,059,100; 5,193,684.

There are a variety of products on the market for females when making a choice between the types of feminine hygiene products to use during menstruation. However, a portable product for proper disposal of used feminine hygiene products is non-existent. This invention fulfills a need for millions of females when it comes to the privacy and convenience of discarding used feminine hygiene products.

In parks, campsites and other public places that do not have specific containers for disposing of used feminine hygiene products, this portable disposable container offers an inexpensive and convenient solution.

This is an opaque color, odor-proof and leak proof portable container made of resin material that opens and closes to insert used feminine hygiene products inside. Once used products are inserted inside said container, the container is then closed and disposed of at the discretion of the user. The advantages of this invention are increased privacy, convenient disposal of used feminine hygiene products, cleaner water systems, and decreased plumbing bills.

This invention relates to an individual portable container made of a resin material used to dispose of used feminine hygiene products. The individual container has two vertical sides that are permanently sealed and the bottom horizontal seam is permanently sealed. The top of the container has a horizontal reclosable opening extending along the longitudinal dimension. The container can be easily hand sealed after the used product is stored inside. The make of the container resembles a reclosable storage bag. However, the entire container is opaque in color and when closed, the container is leak-proof and the inside contents are not visible.

What is claimed is:

1. A method to use an open and reclosable portable container made of resin material that will open allowing used feminine hygiene products that trap secretions or body fluids from the female genital region to be inserted inside said container, said container is closed, said used feminine hygiene products can now be disposed of. Once said used feminine hygiene products are inserted into said container and said container is closed, said container is odor proof and secure. Said container is constructed and colored in a manner so that said used feminine hygiene products stored inside said container are not visible from an outside view of said container.
